(19) United States
(12) Patent Application Publication
(10) Pub. No.: US 2022/0287981 A1
(43) Pub. Date: Sep. 15, 2022

(54) ADHESIVE DRUG DELIVERY MICROPARTICLES AND A PRODUCT COMPRISING THEREOF

(71) Applicant: AMD PHARMA LTD., Tel Aviv (IL)

(72) Inventor: Adel PENHASI, Modiin (IL)

(21) Appl. No.: 17/635,390

(22) PCT Filed: Aug. 16, 2020
(86) PCT No.: PCT/IL2020/050900

§ 371 Date: Feb. 15, 2022

Related U.S. Application Data
(60) Provisional application No. 62/887,816, filed on Aug. 16, 2019.

(51) Int. Cl. A61K 9/50; A61K 9/00

(52) U.S. Cl. CPC A61K 9/5026; A61K 9/0053

(57) ABSTRACT

There is provided herein a microparticle for the administration of a pharmaceutical composition at the upper gastrointestinal tract comprising: a core comprising at least one pharmaceutical composition; at least one excipient a first coating layer comprising a bioadhesive material; optionally, a channel forming agent; a film forming polymer; and a cross-linker wherein said cross-linker interacts with said film forming polymer upon exposure to an aqueous environment.

### FIELD OF THE INVENTION

The present invention relates to the field of pharmaceutics, specifically to an adhesive drug delivery microparticle and to a product comprising thereof specifically aimed at the targeted delivery of pharmaceutical compositions at the upper gastrointestinal tract.

### BACKGROUND OF THE INVENTION

Controlled release systems for drug delivery are often designed to administer drugs in specific areas of the gastrointestinal (GI) tract. Often the challenge with certain drugs is the specific area of absorption, wherein beyond this area, the drug may have little or no absorption into the human body.

The desired absorption rate and extent results in reaching a target blood level of the active substance of the drug, having direct correlation to the safety and effectiveness of the drug.

Some drugs possess a narrow window of absorption in the GI tract, making it extremely difficult to provide effective controlled release compositions containing these drugs, and often resulting in final pharmaceutical compositions that need to he administered several times a day or have a high risk of toxicity or substantial side effects.

Also, beyond the issue of the specific location of the absorption of a drug, there is also importance to the duration of time that the drug is situated in the desired location. With some drugs, especially neuroactive drugs, the patient may suffer from side effects or lower efficacy if blood serum concentrations vary considerably.

Within the entire GI tract, there are specific regions which might be more complicated for drug absorption in comparison to other regions, for example, the stomach which is characterized by an acidic environment and a constant secretion of mucosa on the stomach wails, and for example, the duodenum which is considered the optimal absorption site for several drugs, e.g., due to the secretion of bile, is actually a rather short section within the entire GI tract.

Thus, there is a need for specific compositions and methods for controlling the absorption of pharmaceutical agents transferred through specific regions within the GI tract.

### SUMMARY OF THE INVENTION

According to sonic demonstrative embodiments, there is provided herein a bioadhesive microparticle comprising one or more pharmaceutical compositions, wherein the microparticle comprises one or more pharmaceutical agents which are to be absorbed into the human body via one or more portions of the GI tract, preferably, via the upper GI tract.

According to some demonstrative embodiments, there is provided herein a microparticle for the administration of a pharmaceutical composition at the upper gastrointestinal tract comprising:
a core comprising
   at least one pharmaceutical composition;
   at least one excipient
a first coating layer comprising
   a bioadhesive material;
   optionally, a channel forming agent;
   a film forming polymer; and
   a cross-linker

wherein said cross-linker interacts with said film forming polymer upon exposure to an aqueous environment.

According to some demonstrative embodiments, the core may further comprise a hydrophilic component and a binder.

According to some demonstrative embodiments the first layer may further comprise a channel forming agent.

According to some demonstrative embodiments, the bioadhesive material may be selected from selected from one or more of the following groups of materials:
a. Pulyanionic polymers selected from the group including polycarbophil USP, cross-linked polyacrylic acid polymers such as Carbopol 974P NF (Carbomer Homopolymer Type B), Carbopol 971P NF (Carhomer Homopolymer Type A), Carbopol 934P NF (Carbomer 934P), Carbopol 71 G, Carbopol 980 NF (Carbomer Homopolymer Type C), Carbopol 981 NF, Carbopol 5964 EP, Carbopol 940 NF, Carbopol 941 NF, Carhopol 1342 NF, Carbopol 934 NF, Carbopol ETD 2020 NF, Carhopol Ultrez 10 NF, Carhopol TR-1 NF, Carbopol TR-2 NF, or a combinations thereof.
b. Polycationic polymers selected from the group including cationic starch, cationic polyvinyl alcohol, cationic polysaccharide and/or cationic gum.

According to some demonstrative embodiments, the bioadhesive material may preferably be polycarbophil USP.

According to some demonstrative embodiments, the cross linker may be a composition which allows for the in-situ cross linking of the film forming polymer and is selected from he group including Caffeic acid, tannic acid, chicoric acid, carhodiimide, genipin, Gallic acid, boric acid and sodium borate (borax).

According to some demonstrative embodiments, the channel forming agent may comprise a polymer selected from the group including low molecular weight Polyethylene glycol (PEG), low molecular weight PolyEthylene oxide (PEO), low molecular weight Polyvinylpyrrolidone (PVP), low molecular weight Polyvinyl alcohol (PVA), low molecular weight Sodium Carboxymethyl cellulose (Na-CMC), low molecular weight Hydroxyethyl cellulose (HEC).

According to some demonstrative embodiments, the channel forming agent may be a low molecular weight PVP below 30 Kilo Dalton.

According to some demonstrative embodiments, there is provided herein a product comprising the microparticle of the present invention, wherein the product may comprise microparticles of different diameters or of substantially similar diameter,

According to some demonstrative embodiments, there is provided herein a process for the preparation of the microparticle of the present invention, wherein the core may be prepared by granulation or extrusion.

### DESCRIPTION OF THE DRAWINGS

FIG. 1 depicts a Tensile bioadhesion testing diagram, according to some demonstrative embodiments of the present invention.

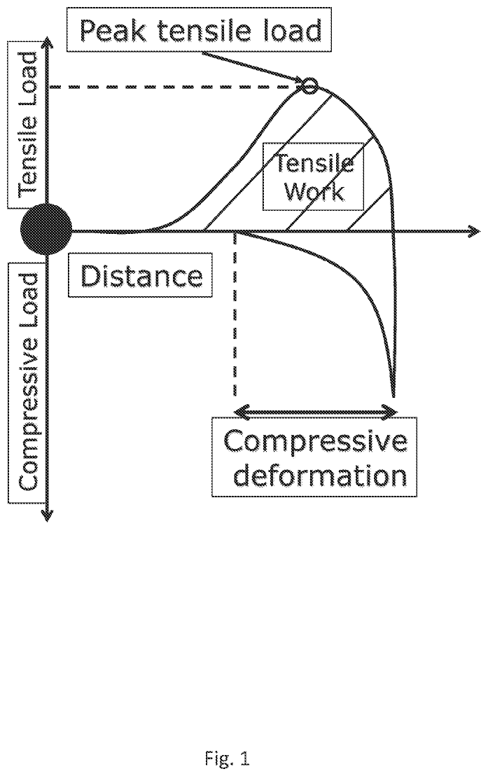

### DETAILED DESCRIPTION OF THE INVENTION

According to some demonstrative embodiments, there is provided herein a bioadhesive microparticle comprising one or more pharmaceutical compositions, wherein the microparticle comprises one or more pharmaceutical agents which are to be absorbed into the human body via one or more portions of the GI tract, preferably, via the upper GI tract.

Drugs are usually classified using the Biopharmaceutical Classification System (BCS), which categorizes pharmaceutical compositions for oral administration into four main classes depending on their solubility and their permeability through the intestinal wall.

According to the BCS, the classifications are as follows:

Class I—High Permeability, High Solubility

Class II—High Permeability, Low Solubility

Class III—Low Permeability, High Solubility

Class IV—Low Permeability, Low Solubility.

According to some embodiments, the term “pharmaceutical composition”, also referred to herein as “the active substance” or “active pharmaceutical ingredient (API)” may include to any suitable drug from Classes I, II, II and IV, Preferably, from Class I and/or II.

According to some embodiments, the pharmaceutical composition may be selected front the group including caffeine, carbamazepine, fluvastatin, Ketoprofen, Metoprolol, Naproxen, Propranolol, Theophylline, Verapamil, Diltiazem, Gabapentin, Levodopa, Divalproex sodium, itraconazole and its relatives, fluoconazole, terconazole, ketoconazole, and saperconazole, griseofulvin and related compounds such as griseoverdin, anti malaria drugs, immune system modulators e.g. cyclosporine, cardiovascular drugs (e.g. digoxin and spironolactone), ibuprofen, danazol, albendazole, clofazimine, acyclovir, carbamazepine, proteins, peptides, polysaccharides, nucleic acids, nucleic acid oligomers, viruses, Neomycin B, Captopril, Atenolol, Valproic Acid, Stavudine, Salbutamol, Acyclovir, Methotrexate, Lamivudine, Ergometrine, Ciprofloxacin, Antiloride, Caspofungin, Clorothiazide, Tobramycin, Cyclosporin, Allopurinol, Acetazolamide, Doxycyclin, Dapsone, Nalidixic Acid, Sulfamethoxazole, Tacrolimus, And Paclitaxel.

According to some embodiments, the bioadhesive microparticle of the present invention may include one or more pharmaceutical agents together with at least one bioadhesive binder and optionally at least one hydrophilic component.

According to some demonstrative embodiments, the term “microparticle”may include any suitable small sized particles including for example, granules, pellets, particulates, grains, spheres and the like.

According to some embodiments, the use of microparticles according to the present embodiments allows for a larger surface area in comparison to a tablet or a large particle, which, for example, directly affects the rate and/or extent of absorption of the API.

According to some embodiments, the term “bioadhesive”, “bioadhesive polymer” or “bioadhesive material” may refer to the bioadhesive compositions disclosed herein, including materials that contain one or more additional components in addition to the bioadhesive polymers and bioadhesive compositions of the invention.

According to some emboli s, bioadhesives may also include blends of one or more bioadhesive polymers.

In some embodiments, the term “bioadhesive polymers” may be used to refer to both compositions where the polymer itself is bioadhesive, as well as compositions where a non- or poorly bioadhesive polymer is combined with a compound that imparts bioadhesive properties to the composition as a whole, as described in detail herein.

A bioadhesive material may generally refer to a material possessing the ability to adhere to a biological surface for an extended period of time. Bioadhesion requires a contact between the bioadhesive material and the receptor surface, such that the bioadhesive material penetrates into the crevice of the surface (e.g. tissue and/or mucus).

According to some demonstrative embodiments, the bioadhesive may include any high molecular weight crosslinked polyacrylic acid polymers. According to some embodiments, such polymers may differ by crosslink density and can be grouped into the following categories.

I. Polymers of acrylic acid crosslinked with allyl sucrose or allyl pentaerythritol (also known as Carbopol homopolymers).

II. Polymers of acrylic acid and C10-C30 alkyl acrylate crosslinked with allyl pentaerythritol (also known as Carbopol copolymers).

III. Carbomer homopolymer or copolymer that contains a block copolymer of polyethylene glycol and a long chain alkyl acid ester (also known as Carbopol interpolymers).

According to some demonstrative embodiments, the bioadhesive may be selected from one or more of the following groups of materials:
1. Polyanionic polymers selected from the group including polycarbophil AA-1 USP, cross-linked polyacrylic acid polymers such as Carbopol 974P NF (Carbomer Homopolymer Type B), Carbopol 971P NF (Carbomer Homopolymer Type A), Carbopol 934P NF (Carbomer 934P), Carbopol 71 G, Carbopol 980 NF (Carbomer Homopolymer Type C), Carbopol 981 NF, Carbopol 5964 EP, Carbopol 940 NF, Carbopol 941 NF, Carbopol 1342 NF, Carbopol 934 NF, Carbopol ETD 2020 NF, Carbopol Ultrez 10 NF, Carbopol TR-1 NF, Carbopol TR-2 NF, or a combinations thereof.
2. Polycationic polymers selected from the group including cationic starch, cationic polyvinyl alcohol, cationic polysaccharide and/or cationic gum. More preferably, the polycationic polymer may he chitosan which is a linear polysaccharide. According to some embodiments, chitosan has a deacetylation degree ranging from 80% to more than 95%. The chitosan may also optionally have a viscosity ranging from 50 mpa to 800 mpa. The chitosan may optionally be carboxymethylchitosan, trimethylchitosan or quaternised chitosan. The cationic starch or polysaccharide may optionally comprise polyglucosamine, one of the components of chitosan. For example, the cationic polymer may optionally be the β-14 polymer of D-glucosamine or the β-1,4 polymer of D-glucosamine and N-acetyl-D-glucosamine. Optional non-limiting examples of cationic gum may include but not limited to cationic guar, and cationic hydroxypropyl guar, and combinations thereof.
In another embodiment, the polycationic polymer may a cationic polyvinyl alcohol non limiting examples of which include a methyl chloride quaternary salt of poly(dimethylamino ethyl acrylate)/polyvinyl alcohol graft copolymer or a methyl sulfate quaternary salt of poly(dimethylamino ethyl acrylate)/polyvinyl alcohol graft copolymer, a polyvinyl alcohol that comprises a pendant quaternary ammonium salt, and combinations thereof, or any other pharmaceutically acceptable cationic polyvinyl alcohol known in the art.

According to some embodiments, the bioadhesive is a high molecular weight acrylic acid polymer crosslinked with divinyl glycol, for example, a Polycarbophil, USP (Noveon®). According to some embodiments, this bioadhesive is able to enhance the delivery of active ingredients to various mucousal membranes.

According to some demonstrative embodiments, the polycarbophil may be present in a concentration of between 40-90% of the formulation, preferably between 60-90%, most preferably around 70%.

According to some demonstrative embodiments, the microparticle may include a channel forming agent, to cause the formation of pores in the microparticle, e.g., to cause the initiation of the extrusion of the pharmaceutical composition from the microparticle into the surrounding environment.

According to some embodiments, the term “channel forming agent” may include any suitable water soluble polymer including, for example low molecular weight Polyethylene glycol (PEG) below 30 KD, low molecular weight PolyEthylene oxide (PEO) below 20 KD, low molecular weight Polyvinylpyrrolidone (PVP) below 30 KD, low molecular weight Polyvinyl alcohol (PVA) below 31 KD, low viscosity grade Sodium Carboxymethyl cellulose (Na-CMC) (such as 7L and 7L2), low viscosity Hydroxyethyl cellulose (HEC) (such as 250 JR or 250 LR), or a combination thereof.

According to some embodiments, the channel forming agent may preferably be a low molecular weight PVP below 30 Kilo Dalton.

According to some demonstrative embodiments, the term “film forming polymer (FFP)” may refer to any suitable polymer capable of forming an in-situ cross-linked film in an aqueous environment, including, for example Hydroxypropyl cellulose (HPC), Hydroxypropyl Methyl Cellulose (HPMC), Polyvinyl alcohol (PVA) and/or hydrolyzed gelatin.

According to some demonstrative embodiments, a cross linker may be used in order to allow for the in-situ cross linking of the film forming polymer

According to some embodiments, the term “cross-linker” as used herein may include a composition selected front the group including Caffeic acid, tannic acid, chicoric acid, carbodiimide and genipin. Gallic acid, boric acid, sodium borate (borax).

According to some embodiments, the cross linker may become active upon contact with an aqueous environment. According to some embodiments, the cross linker may be dissolved in an organic solvent and may not be active when used in coating of the granule, but may become active in the human body due to exposure to water

According to some embodiments, the term “become active” when referred to with regard to the cross linker may mean that an aqueous environment may cause an intermolecular interaction between the cross linker and he film forming polymer, e.g., hindering the dissolution of the film forming polymer by causing cross linkage withing the film forming polymer by the cross linker

The duodenum may be a location to which materials can hardly adhere to, since the villi of the duodenum have a leafy-looking appearance, which is a histologically identifiable structure and the Brunner's glands, which secrete mucus, are found in the duodenum only. The duodenum wall is also composed of a very thin layer of cells that form the muscularis mucosae.

Other areas in the GI tract which are difficult for the adherence of materials may include the stomach, which has an acidic and mucosal environment, and also the jejunum.

According to some demonstrative embodiments, the unique combination of the components of the microparticle of the present invention may be especially beneficial in adherence to the duodenal wall lining, as well as to the Stomach and/or jejunum lining.

According to some embodiments, the microparticle may be an uncoated particle, including the one or more pharmaceutical agents together with at least one bioadhesive binder, at least one hydrophilic component and optionally a binder.

According to some embodiments, the microparticle may be a coated particle, including a core comprising the one or more pharmaceutical agents; optionally a hydrophilic component and optionally a binder;

A first layer comprising a hioadhesive polymer, a hydrophilic component and a film forming polymer.

According to some embodiments, the film forming polymer may include a single polymer or a mixture of film forming polymers, wherein the film forming polymer may be soluble or insoluble (in water or gastric fluids).

According to some demonstrative embodiments, the combination of some important parameters such as the large surface area of the drug delivery system, which is derived from the relatively small particle size of microparticles and the hydrophilic nature of the microparticies, which is driven from the presence of hydrophilic components in the film coating formulation, such as hydrophilic-imparting agent (such as a high molecular weight of polyethylene oxide), the hydrophilic channel-forming agent (channeling agent), and the hydrophilic film-forming polymer, which undergoes in-situ cross-linking by a hydrophilic cross-linker, and finally the hydrophilic bio-adhesive polymer, is specifically beneficial in an immediate and as fast wetting process of the drug delivery system after exposure to the gastric fluid and thus the adherence to the upper parts of the GI tract such as stomach, duodenal and the jejunum region.

According to some demonstrative embodiments, there is provided herein a composition, also referred to herein as a product, comprising a plurality of microparticles according to the present invention.

According to some demonstrative embodiments, the microparticles contained within the composition may have a substantially similar size and/or diameter, for example, to allow for a specific unified release rate.

According to some embodiments, the size of the microparticles determines the rate and/or extent of the absorption of the active substance.

Brunauer-Emmett-Teller (BET) theory aims to explain the physical adsorption of gas molecules on a solid surface and serves as the basis for an important analysis technique for the measurement of the specific surface area of materials.

According to some embodiments, the microparticle size may be in the range between 100 to 1500 microns, preferably between 300 to 1200 microns and most preferably between 500 to 1000 microns.

According to some embodiments, upon exposure to an aqueous environment in the human body, a plurality of pores may be created on the surface of the microparticle of the present invention, for example, due to the dissolution of the channel forming agent(s).

The size, shape, pore volume, pore distribution of the microparticle, may directly affect the surface area and consequentially, the rate and/or extent of release of the pharmaceutical composition from the microparticle.

According to some embodiments, there is provided herein a delivery system containing a variety of microparticles having different sizes, optionally comprising soluble or insoluble polymers, allowing for an immediate release or sustained release or a combination thereof.

### EXAMPLE 1

An experiment was conducted to test the mechanism of action of the delivery system of the present invention, based on microencapsulation of the active materials. The uniqueness of the system is especially manifested by the fact that the bioadhesion properties of the coating layer are mainly activated once the system is exposed to the duodenum pH. The system is then stuck in the duodenum to release the active material, by a controlled manner, directly in the duodenum to provide a long-lasting release to achieve a once daily or a twice daily regime.

In the experiment, multiple formations for testing their bio-adhesion properties in-vitro were prepared and tested using a specific tensile tester {TA.XTPlus Texture Analyzer, Texture Technologies, Scarsdale, N.Y.)) under simulated upper GI tract conditions.

The objective of the experiment wasto assess:

1. Are the formulations bio-adhesive?

2. Does the bio-adhesion force depend on the pH, andandl if so, is it highest at the pH of the duodenum?

3. Are the bio-adhesion properties activated specifically in the duodenal fluid pH or also in the gastric fluid pH?

4. Can the screening of the formulations by the test lead to a specific formulation that is able to present the highest bio-adhesion forces?

5. Can the preparation method of the film samples affect their adhesion features?

Method

During the experiment dozens of different types of film formulations in the form of small disks were prepared. A synthetic mucus, simulating in its chemical composition, the natural mucus existing in the gastro-intestinal system, was used as the substrate. The tests were carried out using a Tensile Tester (T A.XTPlus Texture Analyzer, Texture Technologies, Scarsdale, N.Y.). Prior to the test, the samples were wetted by dropping a certain volume of various buffer solutions, characterizing (in terms of pH) either the duodenum fluid or the stomach fluid.

Results and conclusions front the samples tested:

1. All formulations do exhibit bio-adhesion properties.

2. The bio-adhesion was found to he pH-dependent.

3. The optimal adhesion was specifically found in the pH of the duodenum. In the gastric pH environment, which is characterized by a higher acidity level, the adhesion three was extremely low, indicating a lower capability of adhering onto the stomach wall.

4. Among the different film compositions one could find a specific formulation which was predominantly able to present especially highest adhesion forces.

The unique preparation method of some formulations drastically affected the adhesion ability

| | | | Polycarbophyl | New Formula | Results (mN) | |
|---|---|---|---|---|---|---|
| Sample | | Tannic | AA - 1 | [HPC + Tannic | [Adhesion Test] | Work AVG |
| No. | HPC | Acid | [%] | Acid:Polycarbophyl] | Force Avg | (mN*mm) |
| 1 | X | — | — | 100:0 | 346.964 | 105.238 |
| 1* | X | X | — | 100:0 | 130.259 | 32.228 |
| 1.1 | X | X | 30 | 77:23 | 40.886 | 45.840 |
| 1.2 | X | X | 40 | 70:30 | 37.843 | 33.087 |
| 1.3 | X | X | 50 | 62:38 | 38.656 | 42.061 |
| 1.4 | X | X | 60 | 54:46 | 105.290 | 42.702 |
| 1.5 | X | X | 70 | 30:70 | 184.431 | 87.692 |
| 1.6 | X | X | 80 | 20:80 | 123.638 | 49.013 |
| 1.7 | X | X | 90 | 10:90 | 107.497 | 40.192 |
| 1.8 | X | X | 100 | 0:100 | | |

While this invention has been described in terms of some specific examples, many modifications and variations are possible. It is therefore understood that within the scope of the appended claims, the invention may he realized otherwise than as specifically described.

1. A microparticle for the administration of a pharmaceutical composition at the upper gastrointestinal tract comprising:
a core comprising
at least one pharmaceutical composition,
at least one excipient
a first coating layer comprising
a bioadhesive material;
optionally, a channel forming agent;
a film forming polymer; and
a cross-linker
wherein said cross-linker interacts with said film forming poly upon exposure to an aqueous environment.

2. The microparticle of claim 1, wherein said core further comprises a hydrophilic component and a binder.

3. The microparticle of claim 1, wherein said first layer further comprises channel forming agent.

4. The microparticle of claim wherein said bioadhesive material is selected from selected from one or more of the following groups of materials:
c. Polyanionic polymers selected from the group including polycarbophil USP, cross-linked polyacrylic acid polymers such as Carbopol 974P NF (Carbomer Homopolymer Type B), Carbopol 971P NF (Carbomer Homopolymer Type A), Carhopol 934P NF (Carbomer 934P), Carbopol 71 G, Carbopol 980 NF (Carbomer Homopolymer Type C), Carbopol 981 NF, Carhopol 5964 EP, Carbopol 940 NF, Carhopol 941 NF, Carbopol 1342 NF, Carbopol 934 NF, Carbopol ETD 2020 NF, Carbopol Ultrez 10 NF, Carhopol TR-1 NF, Carhopol TR-2 NF, or a combinations thereof.
d. Polycationic polymers selected front the group including cationic starch, cationic polyvinyl alcohol, cationic polysaccharide and/or cationic gum.

5. The microparticle of claim 4, e ein said bioadhesive material comprises polycarbophil USP.

6. The microparticle of claim 1, wherein said cross linker is a composition which allows for the in-situ cross linking of the film forming polymer and is selected from the group including Caffeic acid, tannic acid, chicoric acid, carbodiimide, genipin, Gallic acid, boric acid and sodium borate (borax).

7. The microparticle of claim 1, wherein said channel forming agent comprises a polymer selected from the group including low molecular weight Polyethylene glycol (PEG), low molecular weight PolyEthylene oxide (PEO), low molecular weight Polyvinylpyrrolidone (PVP), low molecular weight Polyvinyl alcohol (PVA), low molecular weight Sodium Carboxymethyl cellulose (Na-CMC), low molecular weight Hydroxyethyl cellulose (HEC).

8. The rrricroparticle of claim 1, wherein said channel forming agent is a low molecular weight PVP below 30 Kilo Dalton.

9. A product comprising the microparticle of claim 1, wherein said product comprises microparticles of different diameters.

10. A product comprising the microparticle of claim 1, wherein said product comprises microparticles of substantially similar diameter.

11. A process for the preparation of the microparticle of claim 1, wherein said core is prepared by granulation or extrusion.
